(19) United States
(12) Plant Patent Application Publication
(10) Pub. No.: US 2002/0148012 P1
(43) Pub. Date: Oct. 10, 2002

(54) Hybrid tea rose plant named 'RUIVONK'

(76) Inventor: Anton Poijw, De Kwakel (NL)

Correspondence Address: CHRISTIE, PARKER & HALE, LLP, 350 WEST COLORADO BOULEVARD, SUITE 500, PASADENA, CA, 91105, US

(21) Appl. No.: 10/004,187

(22) Filed: Oct. 31, 2001

(30) Foreign Application Priority Data
Jul. 16, 2001 (EP) EU 2000/1930
Apr. 6, 2001 (NL) NL R00 2714

(51) Int. Cl. A01H 5/00

(52) U.S. Cl. PLT/132

(57) ABSTRACT

A new and distinct Hybrid Tea Rose plant having attractive Light Pink flowers.

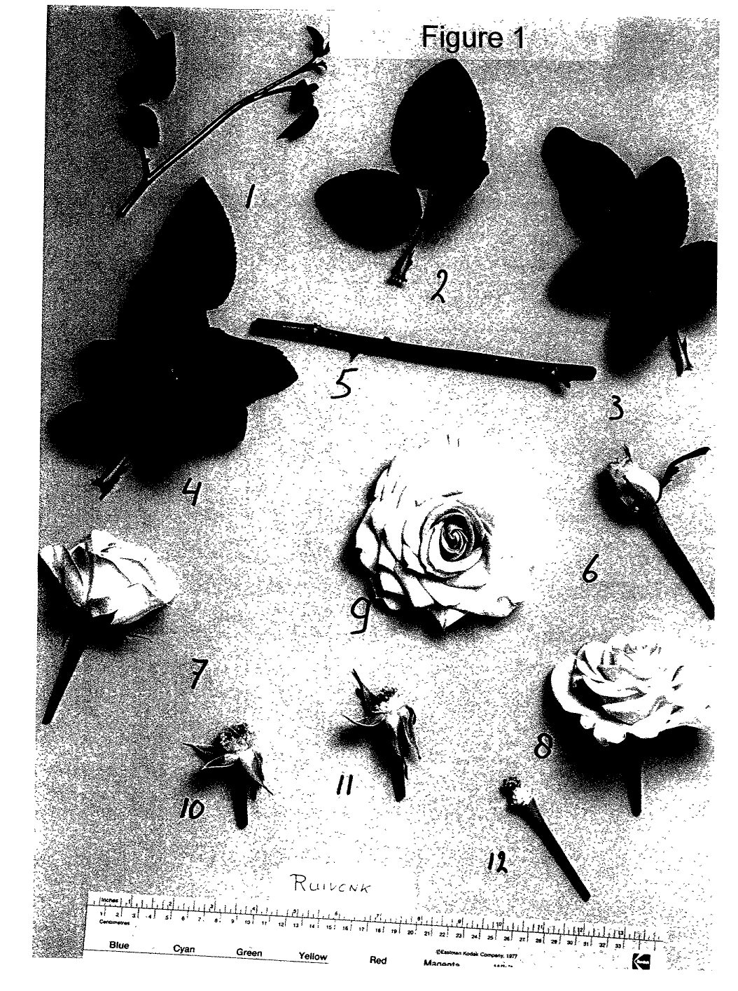

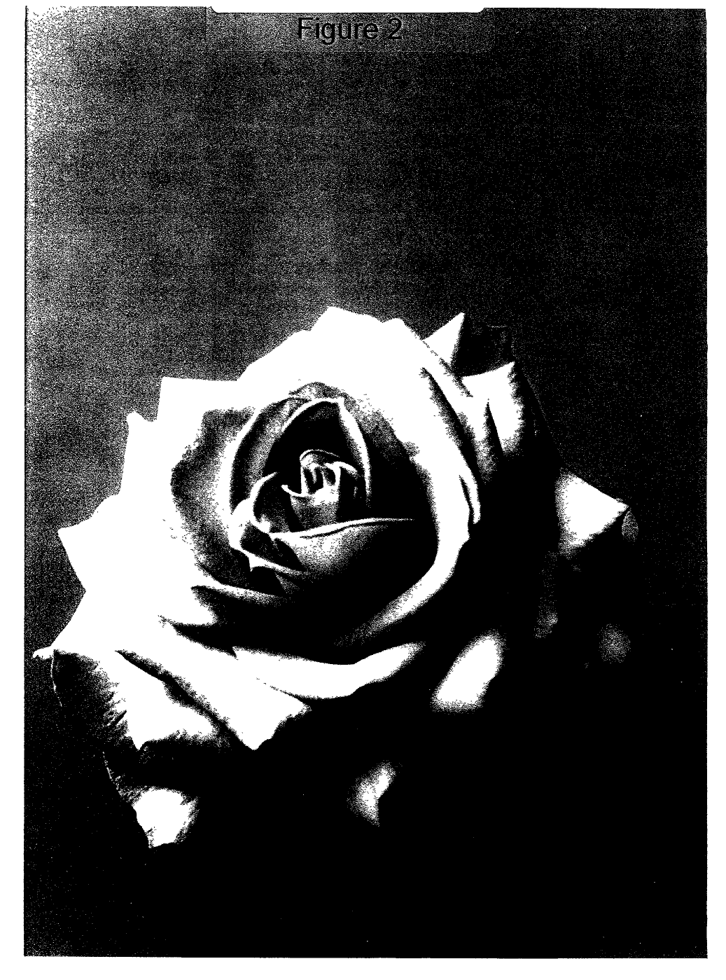

### CROSS-REFERENCE TO RELATED APPLICATION(S)

This Patent Application claims priority of European Patent Application: No. 2000/1930, Certificate No. EU7944, dated Jul. 16, 2001 and Patent Application filed in The Netherlands, Application No. R002714, Certificate No. 19668, dated Apr. 6, 2001.

### BACKGROUND OF THE INVENTION

The present invention relates to a new and distinct variety of rose plant of the Hybrid Tea rose class, which was discovered as a hip on the floor of the hybridizing greenhouse in the nursery of Applicant's Assignee. The varietal denomination of this new rose is named ‘RUIVONK.’ The new variety was grown from a seed in The Netherlands.

### SUMMARY OF THE INVENTION

Among the novel characteristics possessed by this new variety which distinguishes it from all varieties of which I am aware is its attractive flowers and large vase life.

Reproduction by propagation by grafting of the new variety as performed in De Kwakel, The Netherlands shows that the foregoing and other distinguishing characteristics come true to form and are established and transmitted through succeeding propagation.

### BRIEF DESCRIPTION OF ILLUSTRATION(S)

The accompanying illustration shows a typical specimen of the new variety depicted in color as nearly true as is reasonably possible to make the same in a color illustration of this character.

### DESCRIPTION OF THE NEW VARIETY

The following is a detailed description of the new variety with color terminology in accordance with The Royal Horticultural Society Colour Chart (RHSCC) . The terminology used in color description herein refers to plate numbers in the aforementioned color chart. Phenotypic expression may vary with light availability, environmental and cultural conditions. The following observations are made of 9-month old plants grown in a greenhouse in De Kwakel, The Netherlands during September from a rooted cutting.

### FLOWER

Blooming Habit: Continuous

Bud.—Large, width about 4 cm, height about 5 cm. Form: Ovoid Color: When sepals first divide, Light Pink near 56D; with some Green on the margin of the petals, near 145C

Sepals.—3 sepals without projections, 2 sepals with some leaf-like projections, top is pointed, surface looks woolly. Size: Length about 3.5 cm, width at the base about 1 cm Color: Upper Surface: Near 144A Under surface: Near 144A and 176B

Peduncle.—Length: About 8 cm Aspect: Smooth Strength: Erect, heavy Color: Near 143A

Bracts.—None observed.

Bloom

Size.—Average size when fully opened—about 12 cm.

Borne.—Singly.

Form.—At first, top is flat, later becoming flattened convex; at maturity petals are slightly curled.

Petalage.—Number of petals under normal conditions—about 45 . Color (⅓ to ½ open) Outer Petals: Top surface—marginal zone near 62C, middle zone near 155A; Under surface—marginal zone between 62C and 62D middle zone near 155A Inner Petals: Top surface—marginal zone between 62A and 62B, middle zone near 155A; Under surface—marginal zone near 62C, middle zone near 155A

General tonality from a distance.—Light Pink.

Base of Petals (point of attachment).—Yellow, near 9C. Color (Fully Opened)

Outer petals.—Top surface—marginal zone near 62B, middle zone near 155; under surface—marginal zone near 62C, middle zone near 155B.

Inner Petals.—Top surface—marginal zone near 62A, middle zone near 155A; under surface—marginal zone near 62D, middle zone near 155A.

General tonality from a distance.—Light Pink.

Base of petals(point of attachment).—Yellow, near 9C.

Color Change.—As bloom ages, colors lighten from between 62A and 62B to near 62D.

General tonality from a distance.—Light Pink. Texture: Thick Appearance: Smooth Form: About round, cross section about 5 cm Arrangement: Imbricated Petaloids in center: About 10 Persistence: Petals are persistent Fragrance: None Lasting quality On the plant: Extremely long, more than 35 days In the vase: Extremely long, up to 35 days

### REPRODUCTIVE ORGANS

Stamens, Filaments and Anthers

Arrangement.—Regularly arranged around styles, filament length about 1 cm.

Color.—Stamens near 9C, Filaments near 9C, Anthers near 9A.

Pollen.—Color Greyed Yellow Group, near 161D.

Styles.—Bunched, medium length, about 0.8 cm, color Red Group, near 51A.

Stigmas.—Color White Group, near 155

Hips.—Shape: Funnel shaped Size: At full flower length about 1, cm, width at top about 1 cm Color: This variety is not used as a motherplant

### PLANT

Form.—Upright.

Growth.—Uniform branching, vigorous; height attained (first season) about 260 cm.

Foliage.—Compound, 3 to 5 leaflets, sometimes 7 leaflets. Size: Length about 16 cm, width about 11 cm Quantity: About 11 leaves per stem, About 180 stems per m² per year in Holland in a glasshouse

Color

New foliage.—Upper side Purple Green Group near 187A mixed with near 137A; Under side Red Group, near 183B.

Mature foliage.—Upper side Green Group near 138B; Under side Green Group near 138B.

Shape.—Elliptic.

Texture Upper side: Smooth with deep-lying veins Under side: Smooth with high-lying veins

Edge.—Serrated.

Serration.—Serrate.

Petiole.—Top surface near 178A; Under surface between 138B and 178A.

Stipules.—Length about 1.5 cm, edge irregular serrated at both sides.

Auricle.—Absent.

Rachis.—Gully shaped, upright edge, rough.

Resistance to disease.—Susceptible to Blackspot, Mildew and Rust.

### WOOD

New shoots

Bark.—Smooth, Red Green Group near 178A and 146D

Wood.—Near 146B.

Old Wood:

Bark.—Smooth, Green Group near 138A.

Wood.—White Group near 155C.

Flower stem.—About 60 to 90 cm from last cut (including penduncle).

Thorns

Quantity.—About 5 small thorns on basal canes at the basis, few thorns on laterals from basal canes on the 1^{st} half of the stem, no thorns on upper part of the stem.

Form.—Straight to downward curved, length about 0.7 cm, color (when young) Purple Green Group near 183B and near 146D, position irregular.

Prickles.—none observed on main stalks.

Winter Hardiness.—Not tested—indoor variety.

Uniqueness.—Vase life is extremely long, up to 35 days. Very nice color

I claim:

1. A new and distinct Hybrid Tea Rose plant of the variety substantially as shown and described.
